(19) United States
(12) Patent Application Publication
(10) Pub. No.: US 2008/0138804 A1
(43) Pub. Date: Jun. 12, 2008

(54) Buffer composition

(75) Inventor: Philip R. Buzby, Brockton, MA (US)

Correspondence Address: COOLEY GODWARD KRONISH LLP;ATTN: Patent Group, Suite 1100, 777 - 6th Street, NW, WASHINGTON, DC, 20001, US

(73) Assignee: Helicos BioSciences Corporation, Cambridge, MA (US)

(21) Appl. No.: 11/638,257

(22) Filed: Dec. 12, 2006

(51) Int. Cl. C12Q 1/68; C12N 9/22

(52) U.S. Cl. 435/6; 435/199

(57) ABSTRACT

The invention provides compositions for improving the accuracy of a sequencing-by-synthesis reaction by minimizing the incorporation of unlabeled dNTPs.

### BACKGROUND

In a template-dependent nucleic acid synthesis reaction, the sequential addition of nucleotides is catalyzed by a nucleic acid polymerase. Depending on the template and the nature of the reaction, the nucleic acid polymerase may be a DNA polymerase, an RNA polymerase, or a reverse transcriptase.

Single molecule sequencing techniques allow the evaluation of individual nucleic acid molecules in order to identify changes and/or differences affecting genomic function. In single molecule techniques, a nucleic acid fragment is attached to a solid support such that at least a portion of the nucleic acid fragment is individually optically-resolvable. Sequencing is conducted using the fragments as templates. Sequencing events are detected and correlated to the individual strands. See Braslavsky et al., Proc. Natl. Acad. Sci., 100: 3960-64 (2003), incorporated by reference herein.

There is, therefore, a need in the art for compositions and improved methods to increase the accuracy of nucleic acid synthesis reactions, especially in single molecule sequencing.

### SUMMARY

The invention improves the accuracy of nucleic acid sequencing reactions. Compositions and methods of the invention provide a mutated polymerase in a buffer composition that results in increased fidelity in template-dependent nucleic acid synthesis.

In one aspect, the invention provides compositions comprising a polymerase mutated to minimize both 5′-3′ exonuclease activity and 3′-5′ exonuclease activity relative to a corresponding wild-type polymerase; a member selected from magnesium and manganese; an organic solvent; and inorganic pyrophosphatase. This approach uses a mutated or modified polymerase with a novel reaction buffer to minimize or eliminate incorporation of incorrect nucleotides in the synthesis process. In one embodiment, the polymerase has a higher affinity for incorporating labeled nucleotides as opposed to natural nucleotides, thus further reducing or eliminating errors caused by the introduction of “dark” bases in the synthesis process. Buffer compositions of the invention optimize enzymatic cofactors.

In another aspect, the invention provides compositions comprises a polymerase with minimized 5′→3′ and 3′→5′ exonucleoase activity and/or that possesses a higher affinity for a labeled nucleotide relative to, for example, Pyrococcus furiosus (Pfu) DNA polymerase, Pyrococcus woesei (Pwo) DNA polymerase, Thermus thermophilus (Tth) DNA polymerase, Bacillus stearothermophilus DNA polymerase, Thermococcus litoralis (Tli) DNA polymerase, Stoffel fragment, ThermoSequenases, Therminator™, Thermotoga maritima (Tma) DNA polymerase, Thermus aquaticus (Taq) DNA polymerase, DNA polymerase, Pyrococcus kodakaraensis KOD DNA polymerase, JDF-3 DNA polymerase, Pyrococcus GB-D (PGB-D) DNA polymerase, UlTma DNA polymerase, Tgo DNA polymerase, E. coli DNA polymerase I, T7 DNA polymerase, and archaeal DP1I/DP2 DNA polymerase II. In preferred compositions, a non-hydrolyzable nucleoside triphosphates is included. Optionally present in some embodiments are one or more a detergent, a salt, a surfactant, a buffer and a DNA binding protein. In a preferred embodiment, the pH of the buffer is from between about 7.5 to about 9.8. In another preferred embodiment, the buffer optionally includes one or more of dithiothreitol, EDTA, glycerol, spermidine, and/or BSA.

In one aspect, the invention provides methods for sequencing and/or resequencing at least a portion of a nucleic acid, the method comprising the steps of exposing a support-bound nucleic acid duplex, comprising a nucleic acid template hybridized to a nucleic acid primer, to a composition comprising a polymerase with altered exonuclease activity, manganese or magnesium, an inorganic phosphatase, an organic solvent, and a nucleotide comprising an optically-detectable label; determining whether the nucleotide is incorporated into the primer; removing the optically-detectable label from incorporated nucleotide; repeating steps a, b, and c at least once; and compiling a sequence of nucleotides incorporated into the primer.

Sequencing and/or resequencing at least a portion of the complement of the original template increases the accuracy of the sequence information obtained from a given template by providing more than one set of sequence information to compare, for example, to a reference sequence. In another embodiment, the sequence initially obtained can be compared to the sequence obtained from the new template.

Sequencing methods of the invention preferably comprise template/primer duplex attached to a surface. Individual nucleotides added to the surface comprise a detectable label—preferably an optically-detectable label, such as a fluorescent label. Each nucleotide species can comprise a different label, or can comprise the same label. In a preferred embodiment, each duplex is individually optically resolvable in order to facilitate single molecule sequence discrimination. The choice of a surface for attachment of duplex depends upon the detection method employed. Preferred surfaces for methods of the invention include epoxide surfaces and polyelectrolyte multilayer surfaces, such as those described in Braslavsky, et al., supra. Surfaces preferably are deposited on a substrate that is amenable to optical detection of the surface chemistry, such as glass or silica.

Nucleotides useful in the invention include any nucleotide or nucleotide analog, whether naturally-occurring or synthetic. For example, preferred nucleotides include phosphate esters of deoxyadenosine, deoxycytidine, deoxyguanosine, deoxythymidine, adenosine, cytidine, guanosine, and uridine.

Polymerases useful in the invention include any polymerase having one or more of a minimization of both 5′-3′ exonucloease activity and 3′-5′ exonucleoase activity relative to a corresponding wild-type polymerase and optionally possessing a higher affinity for a labeled nucleotide than for a primer nucleic acid and is capable of catalyzing a template-dependent addition of a nucleotide or nucleotide analog to a primer. Depending on the characteristics of the target nucleic acid, a DNA polymerase, an RNA polymerase, a reverse transcriptase, or a mutant or altered form of any of the foregoing can be used. According to one aspect of the invention, a thermophilic polymerase is used, such as, 9°N™, T2™, and a P680G mutant of the Klenow exo⁻ polymerase.

Other aspects and advantages of the invention are provided in the detailed description that follows.

### DETAILED DESCRIPTION

The invention provides methods and compositions for improving nucleic acid sequencing-by-synthesis reactions by providing reaction buffers that mitigate undesirable polymerase activities. A polymerase reaction buffer is provided that removes or adds co-factors that mitigate undesirable activity and increase the accuracy of sequencing reactions. While applicable to bulk sequencing methods, the invention is particularly useful in connection with single molecule sequencing methods.

A composition is provided comprising a polymerase mutated to minimize both 5′-3′ exonuclease activity and 3′-5′ exonuclease activity relative to a corresponding wild-type polymerase. Compositions of the invention further comprise a member selected from magnesium and manganese; an organic solvent; and inorganic pyrophosphatase. One particular advantage of the invention is that it decreases the incorporation of “dark” bases during a sequencing reaction. Inclusion of dark bases leads to sequencing errors, as an unlabeled base will not be included in the sequence. Polymerases useful in the compositions described herein may also comprise a lower catalytic turnover (Kcat), and a lower kd than either a corresponding wild-type polymerase or a polymerase such as, Taq, Pfu, Pwo, Tth, Tli or other known polymerases.

Compositions of the invention may optionally comprise a non-hydrolyzable nucleoside triphosphate. Suitable nucleosides include, for example, dAMP, dCMP, dTMP or dGMP. Modified nucleosides may also be used in the composition of the invention, and include, for example, nucleotide analogs disclosed in co-owned, co-pending U.S. Ser. No. 11/412,569, incorporated by reference herein. Compositions of the invention may also comprise one or more detergents, for example, Triton, Triton X-100, NP-40, Tween 20, and other like detergents. The compositions may also comprise one or more salts, (for example, KCl, NaCl, (NH₄)₂SO₄, MgCl₂, and/or MnCl₂).

and one or more surfactants.

The compositions may also comprise one or more DNA binding proteins, for example, single-stranded binding (SSB) proteins and/or double-stranded binding proteins (DSB). The SSB and DSB proteins aid in reducing the secondary structure of the primer:template. Another protein that may be optionally included in the reaction buffer is an inorganic pyorphosphatase or other enzyme to degrade the pyrophosphate produced during the polymerization reaction. The compositions may also comprise one or more organic solvents, such as dimethyl sulfoxide(DMSO). In certain embodiments, the compositions described herein have a pH from between about 7.5 to about 9.8, or from between about 8 and about 9.5. Further optional components of the compositions of the invention include one or more of dithiothreitol, EDTA, EGTA, glycerol, spermidine, or BSA

In one aspect, described herein are methods of sequencing a nucleic acid comprising a) exposing a support-bound nucleic acid duplex, comprising a nucleic acid template hybridized to a nucleic acid primer, to a buffer composition disclosed herein and a nucleotide comprising an optically-detectable label; b) determining whether said nucleotide is incorporated into a said primer; c) removing said optically-detectable label from incorporated nucleotide; d) repeating steps a, b, and c at least once; and e) compiling a sequence of nucleotides incorporated into said duplex.

Nucleic Acid Polymerases

Nucleic acid polymerases generally useful in the invention include those having reduced processivity. Also preferred are polymerases having minimized 5′-3′ exonucloease activity and 3′-5′ exonucleoase activity relative to a corresponding wild-type polymerase. Also contemplated are polymerases that possesses a higher affinity for a labeled nucleotide than for a non-lableled nucleic acid. The P680G polymerase mutant is an example of a non-processive polymerase. Also useful are DNA polymerases, RNA polymerases, reverse transcriptases, and mutant or altered forms of any of the foregoing. DNA polymerases and their properties are described in detail in, among other places, DNA Replication 2nd edition, Kornberg and Baker, W. H. Freeman, New York, N.Y. (1991). Known DNA polymerases useful in the invention include, but are not limited to, 9°Nm™. DNA polymerase (New England Biolabs), T2, and a P680G mutant of the Klenow exo⁻ polymerase (Tuske et al. (2000) JBC 275(31):23759-23768).

One particular advantage of a polymerase, such as the P680G in single molecule sequencing is that it provides increased accuracy in sequencing templates that contain a stretch of 2 or more bases of the same type, e.g., such as AA or AAA or GGGG or CCCCC. The increased accuracy resulting from the use of a polymerase with reduced processivity ensures that the growing complement strand accurately reflects the sequence of the template strand even in stretches of 2 or more identical bases.

The processivity of a nucleic acid polymerase is modified by one of skill in the art by mutation or other alteration to achieve an polymerase having minimized 5′-3′ and 3′-5′ exonucleoase activity relative to a corresponding wild-type polymerase and possessing a higher affinity for a labeled nucleotide than for a primer nucleic acid.

Exo⁻ Klenow Fragment P680G: A polymerase used in methods described herein is the Exo⁻ Klenow Fragment P680G. Klenow Fragment is an N-terminal truncation of E. coli DNA Polymerase I which retains both polymerase activity and 3′→5′ exonuclease activity, but has lost the 5′→3′ exonuclease activity. Exo⁻ Klenow Fragment has a mutation (D355A, E357A) (SEQ ID NO: 3) at the 3′→5′ exonuclease active site which abolishes the 3′→5′ exonuclease activity of the wild type Klenow fragment, and thus has no exonuclease activity in either direction. The P680G mutant has a glycine in place of a praline at position 680 in the sequence.

### General Considerations

Single Molecule Sequencing Methods

The methods and compositions described herein can be utilized in a wide variety of sequence related applications, including for example, identifying PCR amplicons, RNA fingerprinting, differential display, single-strand conformation polymorphism detection, dideoxy finger printing, restriction maps and restriction fragment length polymorphisms, DNA fingerprinting, genotyping, mutation detection, oligonucleotide ligation assay, sequence specific amplifications, for diagnostics, forensics, identification, developmental biology, molecular medicine, toxicology, and animal breeding.

For example, direct amine attachment is used to attach primer or template to an epoxide surface. The primer or the template can comprise an optically-detectable label in order to determine the location of duplex on the surface. At least a portion of the duplex is optically resolvable from other duplexes on the surface. The surface is preferably passivated with a reagent that occupies portions of the surface that might, absent passivation, fluoresce. Passivation reagents include amines, phosphate, water, sulfates, detergents, and other reagents that reduce native or accumulating surface fluorescence. Sequencing is then accomplished by presenting one or more labeled nucleotide in the presence of a polymerase under conditions that promote complementary base incorporation in the primer. In a preferred embodiment, one base at a time (per cycle) is added and all bases have the same label. There is a wash step after each incorporation cycle, and the label is either neutralized without removal or removed from incorporated nucleotides. After the completion of a predetermined number of cycles of base addition, the linear sequence data for each individual duplex is compiled. Numerous algorithms are available for sequence compilation and alignment as discussed below.

In general, epoxide-coated glass surfaces are used for direct amine attachment of templates, primers, or both. Amine attachment to the termini of template and primer molecules is accomplished using terminal transferase. Primer molecules can be custom-synthesized to hybridize to templates for duplex formation.

A full-cycle is conducted as many times as necessary to complete sequencing of a desired length of template, or resequencing of the desired length of the template complementary sequence. Once the desired number of cycles is complete, the result is a stack of images represented in a computer database. For each spot on the surface that contained an initial individual duplex, there will be a series of light and dark image coordinates, corresponding to whether a base was incorporated in any given cycle. For example, if the template sequence was TACGTACG and nucleotides were presented in the order CAGU(T), then the duplex would be “dark” (i.e., no detectable signal) for the first cycle (presentation of C), but would show signal in the second cycle (presentation of A, which is complementary to the first T in the template sequence). The same duplex would produce signal upon presentation of the G, as that nucleotide is complementary to the next available base in the template, C. Upon the next cycle (presentation of U), the duplex would be dark, as the next base in the template is G. Upon presentation of numerous cycles, the sequence of the template would be built up through the image stack. The sequencing data are then fed into an aligner as described below for resequencing, or are compiled for de novo sequencing as the linear order of nucleotides incorporated into the primer.

The imaging system used in practice of the invention can be any system that provides sufficient illumination of the sequencing surface at a magnification such that single fluorescent molecules can be resolved.

Nucleic Acid Templates

Nucleic acid templates include deoxyribonucleic acid (DNA) and/or ribonucleic acid (RNA). Nucleic acid template molecules can be isolated from a biological sample containing a variety of other components, such as proteins, lipids and non-template nucleic acids. Nucleic acid template molecules can be obtained from any cellular material, obtained from an animal, plant, bacterium, fungus, or any other cellular organism. Biological samples for use in the invention also include viral particles or samples prepared from viral material. Nucleic acid template molecules may be obtained directly from an organism or from a biological sample obtained from an organism, e.g., from blood, urine, cerebrospinal fluid, seminal fluid, saliva, sputum, stool and tissue. Any tissue or body fluid specimen may be used as a source for nucleic acid for use in the invention. Nucleic acid template molecules may also be isolated from cultured cells, such as a primary cell culture or a cell line. The cells or tissues from which template nucleic acids are obtained can be infected with a virus or other intracellular pathogen. A sample can also be total RNA extracted from a biological specimen, a cDNA library, viral, or genomic DNA.

Nucleic acid obtained from biological samples typically is fragmented to produce suitable fragments for analysis. In one embodiment, nucleic acid from a biological sample is fragmented by sonication. Nucleic acid template molecules can be obtained as described in U.S. Patent Application 2002/0190663 A1, published Oct. 9, 2003, the teachings of which are incorporated herein in their entirety. Generally, nucleic acid can be extracted from a biological sample by a variety of techniques such as those described by Maniatis, et al., Molecular Cloning: A Laboratory Manual, Cold Spring Harbor, N.Y., pp. 280-281 (1982). Generally, individual nucleic acid template molecules can be from about 5 bases to about 20 kb. Nucleic acid molecules may be single-stranded, double-stranded, or double-stranded with single-stranded regions (for example, stem- and loop-structures).

A biological sample as described herein may be homogenized or fractionated in the presence of a detergent or surfactant. The concentration of the detergent in the buffer may be about 0.05% to about 10.0%. The concentration of the detergent can be up to an amount where the detergent remains soluble in the solution. In a preferred embodiment, the concentration of the detergent is between 0.1% to about 2%. The detergent, particularly a mild one that is nondenaturing, can act to solubilize the sample. Detergents may be ionic or nonionic. Examples of nonionic detergents include triton, such as the Triton® X series (Triton® X-100 t-Oct-C₆H₄—(OCH₂—CH₂)ₓOH, x=9-10, Triton® X-100R, Triton® X-114 x=7-8), octyl glucoside, polyoxyethylene(9)dodecyl ether, digitonin, IGEPAL® CA630 octylphenyl polyethylene glycol, n-octyl-beta-D-glucopyranoside (betaOG), n-dodecyl-beta, Tween® 20 polyethylene glycol sorbitan monolaurate, Tween® 80 polyethylene glycol sorbitan monooleate, polidocanol, n-dodecyl beta-D-maltoside (DDM), NP40 nonylphenyl polyethylene glycol, C12E8 (octaethylene glycol n-dodecyl monoether), hexaethyleneglycol mono-n-tetradecyl ether (C14EO6), octyl-beta-thioglucopyranoside (octyl thioglucoside, OTG), Emulgen, and polyoxyethylene 10 lauryl ether (C12E10). Examples of ionic detergents (anionic or cationic) include deoxycholate, sodium dodecyl sulfate (SDS), N-lauroylsarcosine, and cetyltrimethylammoniumbromide (CTAB). A zwitterionic reagent may also be used in the purification schemes of the present invention, such as Chaps, zwitterion 3-14, and 3-[(3-cholamidopropyl)dimethylammonio]-1-propanesulf-onate. It is contemplated also that urea may be added with or without another detergent or surfactant.

Lysis or homogenization solutions may further contain other agents, such as reducing agents. Examples of such reducing agents include dithiothreitol (DTT), β-mercaptoethanol, DTE, GSH, cysteine, cysteamine, tricarboxyethyl phosphine (TCEP), or salts of sulfurous acid.

Nucleotides

Nucleotides useful in the invention include any nucleotide or nucleotide analog, whether naturally-occurring or synthetic. For example, preferred nucleotides include phosphate esters of deoxyadenosine, deoxycytidine, deoxyguanosine, deoxythymidine, adenosine, cytidine, guanosine, and uridine. Other nucleotides useful in the invention comprise an adenine, cytosine, guanine, thymine base, a xanthine or hypoxanthine; 5-bromouracil, 2-aminopurine, deoxyinosine, or methylated cytosine, such as 5-methylcytosine, and N4-methoxydeoxycytosine. Also included are bases of polynucleotide mimetics, such as methylated nucleic acids, e.g., 2′-O-methRNA, peptide nucleic acids, modified peptide nucleic acids, locked nucleic acids and any other structural moiety that can act substantially like a nucleotide or base, for example, by exhibiting base-complementarity with one or more bases that occur in DNA or RNA and/or being capable of base-complementary incorporation, and includes chain-terminating analogs. Other useful nucleotide analogues include, for example, those disclosed in U.S. Ser. No. 11/412,569. A nucleotide corresponds to a specific nucleotide species if they share base-complementarity with respect to at least one base.

Nucleotides for nucleic acid sequencing according to the invention preferably comprise a detectable label that is directly or indirectly detectable. Preferred labels include optically-detectable labels, such as fluorescent labels. Examples of fluorescent labels include, but are not limited to, 4-acetamido-4′-isothiocyanatostilbene-2,2′disulfonic acid; acridine and derivatives: acridine, acridine isothiocyanate; 5-(2′-aminoethyl)aminonaphthalene-1-sulfonic acid (EDANS); 4-amino-N-[3-vinylsulfonyl)phenyl]naphthalimide-3,5 disulfonate; N-(4-anilino-1-naphthyl)maleimide; anthranilamide; BODIPY; Brilliant Yellow; coumarin and derivatives; coumarin, 7-amino-4-methylcoumarin (AMC, Coumarin 120), 7-amino-4-trifluoromethylcouluarin (Coumaran 151); cyanine dyes; cyanosine; 4,6-diaminidino-2-phenylindole (DAPI); 5′5″-dibromopyrogallol-sulfonaphthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansylchloride); 4-dimethylaminophenylazophenyl-4′-isothiocyanate (DABITC); eosin and derivatives; eosin, eosin isothiocyanate, erythrosin and derivatives; erythrosin B, erythrosin, isothiocyanate; ethidium; fluorescein and derivatives; 5-carboxyfluorescein (FAM), 5-(4,6-dichlorotriazin-2-yl)aminofluorescein (DTAF), 2′7′-dimethoxy-4′5′-dichloro-6-carboxyfluorescein, fluorescein, fluorescein isothiocyanate, QFITC, (XRITC); fluorescamine; IR144; IR1446; Malachite Green isothiocyanate; 4-methylumbelliferoneortho cresolphthalein; nitrotyrosine; pararosaniline; Phenol Red; B-phycoerythrin; o-phthaldialdehyde; pyrene and derivatives: pyrene, pyrene butyrate, succinimidyl 1-pyrene; butyrate quantum dots; Reactive Red 4 (Cibacron™ Brilliant Red 3B-A) rhodamine and derivatives: 6-carboxy-X-rhodamine (ROX), 6-carboxyrhodamine (R6G), lissamine rhodamine B sulfonyl chloride rhodamine (Rhod), rhodamine B, rhodamine 123, rhodamine X isothiocyanate, sulforhodamine B, sulforhodamine 101, sulfonyl chloride derivative of sulforhodamine 101 (Texas Red); N,N,N′,N′tetramethyl-6-carboxyrhodamine (TAMRA); tetramethyl rhodamine; tetramethyl rhodamine isothiocyanate (TRITC); riboflavin; rosolic acid; terbium chelate derivatives; Cy3; Cy5; Cy5.5; Cy7; IRD 700; IRD 800; La Jolta Blue; phthalo cyanine; and naphthalo cyanine. Preferred fluorescent labels are cyanine-3 and cyanine-5. Labels other than fluorescent labels are contemplated by the invention, including other optically-detectable labels.

Surfaces

In a preferred embodiment, nucleic acid template molecules are attached to a substrate (also referred to herein as a surface) and subjected to analysis by sequencing as taught herein. Nucleic acid template molecules are attached to the surface such that the template/primer duplexes are individually optically resolvable. Substrates for use in the invention can be two- or three-dimensional and can comprise a planar surface (e.g., a glass slide) or can be shaped. A substrate can include glass (e.g., controlled pore glass (CPG), quartz, plastic (such as polystyrene (low cross-linked and high cross-linked polystyrene), polycarbonate, polypropylene and poly(methymethacrylate)), acrylic copolymer, polyamide, silicon, metal (e.g., alkanethiolate-derivatized gold), cellulose, nylon, latex, dextran, gel matrix (e.g., silica gel), polyacrolein, or composites.

Suitable three-dimensional substrates include, for example, spheres, microparticles, beads, membranes, slides, plates, micromachined chips, tubes (e.g., capillary tubes), microwells, microfluidic devices, channels, filters, or any other structure suitable for anchoring a nucleic acid. Substrates can include planar arrays or matrices capable of having regions that include populations of template nucleic acids or primers. Examples include nucleoside-derivatized CPG and polystyrene slides; derivatized magnetic slides; polystyrene grafted with polyethylene glycol, and the like.

In one embodiment, a substrate is coated to allow optimum optical processing and nucleic acid attachment. Substrates for use in the invention can also be treated to reduce background. Exemplary coatings include epoxides, and derivatized epoxides (e.g., with a binding molecule, such as streptavidin). The surface can also be treated to improve the positioning of attached nucleic acids (e.g., nucleic acid template molecules, primers, or template molecule/primer duplexes) for analysis. As such, a surface according to the invention can be treated with one or more charge layers (e.g., a negative charge) to repel a charged molecule (e.g., a negatively charged labeled nucleotide). For example, a substrate according to the invention can be treated with polyallylamine followed by polyacrylic acid to form a polyelectrolyte multilayer. The carboxyl groups of the polyacrylic acid layer are negatively charged and thus repel negatively charged labeled nucleotides, improving the positioning of the label for detection. Coatings or films applied to the substrate should be able to withstand subsequent treatment steps (e.g., photoexposure, boiling, baking, soaking in warm detergent-containing liquids, and the like) without substantial degradation or disassociation from the substrate.

Examples of substrate coatings include, vapor phase coatings of 3-aminopropyltrimethoxysilane, as applied to glass slide products, for example, from Molecular Dynamics, Sunnyvale, Calif. In addition, generally, hydrophobic substrate coatings and films aid in the uniform distribution of hydrophilic molecules on the substrate surfaces. Importantly, in those embodiments of the invention that employ substrate coatings or films, the coatings or films that are substantially non-interfering with primer extension and detection steps are preferred. Additionally, it is preferable that any coatings or films applied to the substrates either increase template molecule binding to the substrate or, at least, do not substantially impair template binding.

Various methods can be used to anchor or immobilize the primer to the surface of the substrate. The immobilization can be achieved through direct or indirect bonding to the surface. The bonding can be by covalent linkage. See, Joos et al., Analytical Biochemistry 247:96-101, 1997; Oroskar et al., Clin. Chem. 42:1547-1555, 1996; and Khandjian, Mol. Bio. Rep. 11:107-115, 1986. A preferred attachment is direct amine bonding of a terminal nucleotide of the template or the primer to an epoxide integrated on the surface. The bonding also can be through non-covalent linkage. For example, biotin-streptavidin (Taylor et al., J. Phys. D. Appl. Phys. 24:1443, 1991) and digoxigenin with anti-digoxigenin (Smith et al., Science 253:1122, 1992) are common tools for anchoring nucleic acids to surfaces and parallels. Alternatively, the attachment can be achieved by anchoring a hydrophobic chain into a lipid monolayer or bilayer. Other methods for known in the art for attaching nucleic acid molecules to substrates also can be used.

Detection

Any detection method may be used that is suitable for the type of label employed. Thus, exemplary detection methods include radioactive detection, optical absorbance detection, e.g., UV-visible absorbance detection, optical emission detection, e.g., fluorescence or chemiluminescence. For example, extended primers can be detected on a substrate by scanning all or portions of each substrate simultaneously or serially, depending on the scanning method used. For fluorescence labeling, selected regions on a substrate may be serially scanned one-by-one or row-by-row using a fluorescence microscope apparatus, such as described in Fodor (U.S. Pat. No. 5,445,934) and Mathies et al. (U.S. Pat. No. 5,091,652). Devices capable of sensing fluorescence from a single molecule include scanning tunneling microscope (siM) and the atomic force microscope (AFM). Hybridization patterns may also be scanned using a CCD camera (e.g., Model TE/CCD512SF, Princeton Instruments, Trenton, N.J.) with suitable optics (Ploem, in Fluorescent and Luminescent Probes for Biological Activity Mason, T. G. Ed., Academic Press, Landon, pp. 1-11 (1993), such as described in Yershov et al., Proc. Natl. Aca. Sci. 93:4913 (1996), or may be imaged by TV monitoring. For radioactive signals, a phosphorimager device can be used (Johnston et al., Electrophoresis, 13:566, 1990; Drmanac et al., Electrophoresis, 13:566, 1992; 1993). Other commercial suppliers of imaging instruments include General Scanning Inc., (Watertown, Mass. on the World Wide Web at genscan.com), Genix Technologies (Waterloo, Ontario, Canada; on the World Wide Web at confocal.com), and Applied Precision Inc. Such detection methods are particularly useful to achieve simultaneous scanning of multiple attached template nucleic acids.

A number of approaches can be used to detect incorporation of fluorescently-labeled nucleotides into a single nucleic acid molecule. Optical setups include near-field scanning microscopy, far-field confocal microscopy, wide-field epi-illumination, light scattering, dark field microscopy, photoconversion, single and/or multiphoton excitation, spectral wavelength discrimination, fluorophore identification, evanescent wave illumination, and total internal reflection fluorescence (TIRF) microscopy. In general, certain methods involve detection of laser-activated fluorescence using a microscope equipped with a camera. Suitable photon detection systems include, but are not limited to, photodiodes and intensified CCD cameras. For example, an intensified charge couple device (ICCD) camera can be used. The use of an ICCD camera to image individual fluorescent dye molecules in a fluid near a surface provides numerous advantages. For example, with an ICCD optical setup, it is possible to acquire a sequence of images (movies) of fluorophores.

Some embodiments of the present invention use TIRF microscopy for two-dimensional imaging. TIRF microscopy uses totally internally reflected excitation light and is well known in the art. See, e.g., the World Wide Web at nikon-instruments.jp/eng/page/products/tirf.aspx. In certain embodiments, detection is carried out using evanescent wave illumination and total internal reflection fluorescence microscopy. An evanescent light field can be set up at the surface, for example, to image fluorescently-labeled nucleic acid molecules. When a laser beam is totally reflected at the interface between a liquid and a solid substrate (e.g., a glass), the excitation light beam penetrates only a short distance into the liquid. The optical field does not end abruptly at the reflective interface, but its intensity falls off exponentially with distance. This surface electromagnetic field, called the “evanescent wave”, can selectively excite fluorescent molecules in the liquid near the interface. The thin evanescent optical field at the interface provides low background and facilitates the detection of single molecules with high signal-to-noise ratio at visible wavelengths.

The evanescent field also can image fluorescently-labeled nucleotides upon their incorporation into the attached template/primer complex in the presence of a polymerase. Total internal reflectance fluorescence microscopy is then used to visualize the attached template/primer duplex and/or the incorporated nucleotides with single molecule resolution.

Analysis

Alignment and/or compilation of sequence results obtained from the image stacks produced as generally described above utilizes look-up tables that take into account possible sequences changes (due, e.g., to errors, mutations, etc.). Essentially, sequencing results obtained as described herein are compared to a look-up type table that contains all possible reference sequences plus 1 or 2 base errors.

In resequencing, a preferred embodiment for sequence alignment compares sequences obtained to a database of reference sequences of the same length, or within 1 or 2 bases of the same length, from the initially obtained sequence or the target sequence contained in a look-up table format. In a preferred embodiment, the look-up table contains exact matches with respect to the reference sequence and sequences of the prescribed length or lengths that have one or two errors (e.g., 9-mers with all possible 1-base or 2-base errors). The obtained sequences are then matched to the sequences on the look-up table and given a score that reflects the uniqueness of the match to sequence(s) in the table. The obtained sequences are then aligned to the reference sequence based upon the position at which the obtained sequence best matches a portion of the reference sequence. More detail on the alignment process is provided below in the Example.

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein.

We claim:

1. A composition comprising:
a polymerase mutated to minimize both 5′-3′ exonucloease activity and 3′-5′ exonucleoase activity relative to a corresponding wild-type polymerase and that possesses a higher affinity for a labeled nucleotide than for a primer nucleic acid;
a member selected from magnesium and manganese;
an organic solvent; and
inorganic pyrophosphatase.

2. The composition of claim 1, further comprising a non-hydrolyzable nucleoside triphosphate.

3. The composition of claim 1, further comprising one or more members of the group consisting of a detergent, a salt, a surfactant, a buffer and a DNA binding protein.

4. The composition of claim 2, wherein the detergent is Triton, Triton X-100, NP-40, or Tween 20.

5. The composition of claim 3, wherein the salt is selected from the group consisting of KCl, NaCl, (NH₄)₂SO₄, MgCl₂, and MnCl₂.

6. The composition of claim 3, wherein the buffer comprises Tris-HCl.

7. The composition of claim 1, wherein pH is from between about 7.5 to about 9.8.

8. The composition of claim 1, wherein the organic solvent is DMSO.

9. The composition of claim 2, wherein the non-hydrolysable nucleoside triphosphate is dAMP, dCMP, dTMP or dGMP

10. The composition of claim 1, further comprising one or more of dithiothreitol, EDTA, glycerol, spermidine, and BSA.

11. A method for sequencing a nucleic acid, the method comprising the steps of:
a. exposing a support-bound nucleic acid duplex, comprising a nucleic acid template hybridized to a nucleic acid primer, to a composition according to claim 1 and a nucleotide comprising an optically-detectable label;
b. determining whether said nucleotide is incorporated into a said primer;
c. removing said optically-detectable label from incorporated nucleotide;
d. repeating steps a, b, and c at least once; and
e. compiling a sequence of nucleotides incorporated into said duplex.
